(19) United States
(12) Patent Application Publication
(10) Pub. No.: US 2018/0333423 A1
(43) Pub. Date: Nov. 22, 2018

(54) METHODS OF TREATING TESTOSTERONE DEFICIENCY

(71) Applicant: Clarus Therapeutics, Inc., Northbrook, IL (US)

(72) Inventors: Robert E. DUDLEY, Murfreesboro, TN (US); Theodore DANOFF, Philadelphia, PA (US); James A. LONGSTRETH, Mundelein, IL (US)

(21) Appl. No.: 15/984,028

(22) Filed: May 18, 2018

Related U.S. Application Data
(60) Provisional application No. 62/508,195, filed on May 18, 2017.

(51) Int. Cl. A61K 31/568; A61P 5/26; G01N 33/74; A61K 9/00; A61K 47/44; A61K 47/12; A61K 47/10; A61K 47/14

(52) U.S. Cl. CPC A61K 31/568; A61P 5/26; G01N 33/743; A61K 47/14; A61K 47/44; A61K 47/12; A61K 47/10; A61K 9/0053

(57) ABSTRACT

Methods of treating a testosterone deficiency or its symptoms with a pharmaceutical formulation of testosterone esters are provided. The methods are designed to provide optimum plasma testosterone levels over an extended period.

This application claims priority to U.S. provisional application No. 62/508,195 filed May 18, 2017, the disclosure of which is incorporated by reference herein in its entirety.

The present invention relates to treatments for testosterone deficiency and methods utilizing oral formulations of testosterone esters that optimize the plasma testosterone concentration during chronic treatment.

Testosterone (T) is a primary androgenic hormone produced in the interstitial cells of the testes and is responsible for normal growth, development and maintenance of male sex organs and secondary sex characteristics (e.g., deepening voice, muscular development, facial hair, etc.). Throughout adult life, testosterone is necessary for proper functioning of the testes and its accessory structures, prostate and seminal vesicle; for sense of well-being; and for maintenance of libido, erectile potency.

Testosterone deficiency—insufficient secretion of T characterized by low total T concentrations—can give rise to medical conditions (e.g., hypogonadism) in males. Symptoms associated with male hypogonadism include impotence and decreased sexual desire, fatigue and loss of energy, mood depression, regression of secondary sexual characteristics, decreased muscle mass, and increased fat mass. Furthermore, hypogonadism in men is a risk factor for osteoporosis, metabolic syndrome, type II diabetes and cardiovascular disease.

Various testosterone replacement therapies are commercially available for the treatment of male hypogonadism. Pharmaceutical preparations include both testosterone and testosterone derivatives in the form of intramuscular injections, implants, oral tablets of alkylated T (e.g., methyltestosterone), topical gels, topical patches, or an intranasal gel. All of the current T therapies, however, fail to adequately provide an easy and clinically effective method of delivering T. For example, intramuscular injections are painful and are associated with significant fluctuations in circulating T levels between doses; T patches are generally associated with levels of T in the lower range of normal (i.e., clinically ineffective) and often cause substantial skin irritation; and T gels have been associated with unsafe transfer of T from the user to women and children. As well, the sole “approved” oral T therapy in the U.S., methyltestosterone, is associated with a significant occurrence of liver toxicity while oral TU preparations available in many countries fail to yield effective T concentrations unless multiple doses consisting of many capsules are taken each day. Over time, therefore, the current methods of treating testosterone deficiency suffer from poor compliance and thus unsatisfactory treatment of men with low T. For example, in a recently published study, patient adherence to topical T replacement therapy at 6 months was only 34.7% and by 12 months, only 15.4% of patients continued on topical T therapy (Medication Adherence and Treatment Patterns for Hypogonadal Patients Treated with Topical Testosterone Therapy: A Retrospective Medical Claims Analysis. Michael Jay Schoenfeld, Emily Shortridge, Zhanglin Cui and David Muram, Journal of Sexual Medicine March 2013).

Testosterone and its short-chain aliphatic esters (prodrugs of testosterone) are poorly bioavailable—owing to extensive first pass intestinal and hepatic metabolism. On the other hand, long-chain aliphatic esters of testosterone having 16 or more carbons, although bioavailable, undergo very slow hydrolysis of the ester bond, in vivo, and thus do not release effective amounts of testosterone to achieve clinical efficacy. Thus, with testosterone aliphatic ester prodrugs an optimum chain length is required for improved bioavailability, in vivo hydrolysis, and testosterone release. For example, testosterone and testosterone esters with aliphatic side chains of less than 10 carbons in length are primarily absorbed via the portal circulation resulting in substantial, if not total, first pass metabolism. Fatty acid esters of medium and long chain fatty acids (i.e., 11 or more carbons) can be absorbed by intestinal lymphatics, but the longer the fatty acid chain length, the slower the rate and extent of hydrolysis of the ester by in vivo esterases to liberate testosterone thus resulting in poor (i.e., clinically ineffective) pharmacological activity.

Other than selection of a testosterone ester with an optimum side chain length, the formulation of the resulting testosterone ester presents unique challenges. The gastrointestinal environment is decidedly aqueous in nature, which requires that drugs must be solubilized for absorption. However, testosterone and particularly its esters are insoluble in water and aqueous media, and even if the T or T ester is solubilized initially in the formulation, the formulation must be able to maintain the drug in a soluble or dispersed form in the intestine without precipitation or, otherwise, coming out of solution. Simulated intestinal fluids are frequently employed to optimize the formulation in vitro and correlate the in vitro behavior to in vivo performance as reflected in the pharmacokinetic parameters. Furthermore, an oral T formulation must, effectively release T or T ester according to a desired release profile. Hence, an effective formulation of T or T ester must balance good solubility with optimum release and satisfaction of a targeted plasma concentration profile and therapeutic index requirements for testosterone therapy.

Additionally, non-specific esterases in blood may lead to hydrolysis of testosterone undecanoate (TU) (and its metabolite DHTU) during the blood collection procedure used to collect serum or plasma for evaluating T, dihydrotestosterone (DHT), TU and dihydrotestosterone undecanoate (DHTU) concentrations. If the extent of hydrolysis ex vivo is significant (e.g., at high concentrations of circulating TU or DHTU), then serum or plasma values of T, DHT, TU and DHTU may not accurately reflect the true in vivo concentrations of these compounds (e.g., T and DHT values may be artificially high due to ex vivo conversion of TU to T or DHTU to DHT). It has previously been shown that the collection of blood in sodium fluoride (NaF) containing tubes (so called ‘gray tops’ in a clinical setting) interfered with the measurement of serum T with a 20% decrease in the measured serum T levels (Wang, et al, 2008). Recent repeat experiments in the Endocrine and Metabolic Research Lab at Los Angeles Biomedical Research Institute (LA Biomed) revealed that addition of TU in excess of 500 ng/ml resulted in about a 20 to 25% increase in measured T concentrations when collected in standard blood collection tubes (so called ‘red top’ tubes; i.e., plain tube without additives), indicating ex vivo conversion of TU to T. In preliminary experiments, LA Biomed performed in vitro experiments by collecting blood into different tubes (i.e., Plain tubes, NaF+EDTA tubes, and NaF+oxalate tubes) spiked with 0, 300 or 600 ng/ml of TU. The experiments demonstrate that the blood collected in the NaF+oxalate and NaF+EDTA tubes and kept at 4° C. for 30 minutes showed minimal increases in the measured T levels after spiking with TU, indicating that the TU was only minimally degraded into T. This was not true in other tubes that did not contain NaF. In addition, the measured T concentrations in fluoride-containing tubes decreased by about 12 to 25%. Lachance et al published an article in 2015 that concluded T must be analyzed in enzyme-inhibited (i.e., NaF containing) plasma or serum when TU is administered to subjects in an oral form.

For these reasons, among others, no oral formulation of testosterone or testosterone esters has been approved by the United States Food and Drug Administration (FDA) to date. In fact, the only oral testosterone product ever approved to date by the FDA is methyltestosterone (in which a methyl group is covalently bound to the testosterone “nucleus” at the C-17 position to inhibit hepatic metabolism; note, also, that methyltestosterone is a chemical derivative and not a prodrug of testosterone) and this approval occurred several decades ago. Unfortunately, use of methyltestosterone has been associated with a significant incidence of liver toxicity, and it is rarely prescribed to treat men with low testosterone.

As noted above, fatty acid esters of testosterone provide yet another mode of potential delivery of testosterone to the body (i.e., as a “prodrug”). Once absorbed, testosterone can be liberated from its ester via the action of non-specific tissue and blood esterases. Furthermore, by increasing the relative hydrophobicity of the testosterone moiety and the lipophilicity of the resulting molecule as determined by its n-octanol-water partition coefficient (log P) value, such prodrugs will be absorbed, primarily via the intestinal lymphatics, thus reducing first-pass metabolism by the liver. In general, lipophilic compounds having a log P value of at least 5 and oil (triglyceride) solubility of at least 50 mg/mL are transported primarily via the lymphatic system.

Despite their promise, prodrugs of testosterone, including testosterone esters, have not been formulated in a manner to achieve effective and sustained plasma testosterone levels at eugonadal levels (i.e., average serum/plasma T concentration falling in the range of about 300-1100 ng/dL when blood collected into plain (i.e., red top) tubes; average T concentration in plasma isolated from blood collected in NaF-EDTA (i.e., gray top) tubes falling in the range of about 252-907 ng/dL). In fact, an orally administered pharmaceutical preparation of a testosterone prodrug, including testosterone esters, has yet to be approved by the FDA.

Thus, there remains a need for an oral formulation of a testosterone ester, which provides optimum plasma testosterone levels that are clinically effective to treat hypogonadal men (i.e., those with a serum/plasma T concentration of <300 ng/dL when blood collected into a plain tube) over an extended period.

Thus, in various embodiments, the present invention provides a method of treating chronic testosterone deficiency in a subject in need thereof comprising the steps of:
a) administering daily to the subject a defined dose of an oral pharmaceutical composition comprising a testosterone ester solubilized in a carrier comprising at least one lipophilic surfactant and at least one hydrophilic surfactant;
b) measuring the circulating testosterone concentration in the subject from which blood is collected into tube containing NaF; and
c) increasing the dose of testosterone ester administered in step a. when the measured plasma testosterone C_{avg} in the subject is less than about 350 ng/dL, decreasing each dose of testosterone ester administered in step a. when the plasma testosterone C_{avg} (as estimated on the basis of a single sample of blood collected about 3 to 6 hours after oral TU) in the subject is greater than about 800 ng/dL, and maintaining each dose of testosterone ester administered in step a. when the measured plasma testosterone C_{avg} in the subject is between about 350 ng/dL and about 800 ng/dL.

In certain embodiments, the steps a.-c. are repeated until the plasma testosterone concentration in the subject is between about 350 and about 800 ng/dL.

In an embodiment, said circulating testosterone concentration is measured in plasma.

In an embodiment, said circulating testosterone concentration is measured in serum.

In an embodiment, said blood is collected into tube containing NaF-EDTA.

In an embodiment, said blood is collected into tube containing NaF-oxalate.

In an embodiment, said blood is drawn 3-5 hours after said administration of said dose.

In an embodiment, said blood is drawn 4-6 hours after said administration of said dose.

In an embodiment, said blood is drawn at least 7 days after starting treatment and following dose adjustment.

In an embodiment, said plasma is NaF-containing plasma.

In various embodiments, the testosterone ester is a short-chain (C₂-C₆) or a medium-chain (C₇-C₁₃) fatty acid ester. In certain embodiments, the testosterone ester is a medium-chain fatty acid ester selected from the group consisting of testosterone cypionate, testosterone octanoate, testosterone enanthate, testosterone decanoate, and testosterone undecanoate (TU), testosterone tridecanoate (TT), or combinations thereof.

In particular embodiments, the testosterone ester is testosterone undecanoate.

In another embodiment, said testosterone ester is testosterone tridecanoate.

In various embodiments, the initial dose of testosterone ester in the oral pharmaceutical composition is equivalent to about 150 mg of testosterone. In certain embodiments, the oral pharmaceutical composition comprises testosterone undecanoate. In particular embodiments, the oral pharmaceutical composition administered comprises about 237 mg of testosterone undecanoate that equates to 150 mg of testosterone.

In various embodiments, the initial dose of testosterone ester in the oral pharmaceutical composition is equivalent to about 200 mg of testosterone per dose. In certain embodiments, the oral pharmaceutical composition comprises testosterone undecanoate. In particular embodiments, the oral pharmaceutical composition administered comprises about 316 mg of testosterone undecanoate that equates to 200 mg testosterone per dose.

In various embodiments, the initial dose of testosterone ester in the oral pharmaceutical composition is equivalent to about 250 mg of testosterone per dose. In certain embodiments, the oral pharmaceutical composition comprises testosterone undecanoate. In particular embodiments, the oral pharmaceutical composition administered comprises about 396 mg of testosterone undecanoate that equates to 250 mg testosterone per dose.

In various embodiments, the initial dose of testosterone ester in the oral pharmaceutical composition is equivalent to about 125 mg of testosterone per dose. In certain embodiments, the oral pharmaceutical composition comprises testosterone undecanoate. In particular embodiments, the oral pharmaceutical composition administered comprises about 198 mg of testosterone undecanoate that equates to 125 mg testosterone per dose.

In various embodiments, the initial dose of testosterone ester in the oral pharmaceutical composition is equivalent to about 100 mg of testosterone per dose. In certain embodiments, the oral pharmaceutical composition comprises testosterone undecanoate. In particular embodiments, the oral pharmaceutical composition administered comprises about 158 mg of testosterone undecanoate that equates to 100 mg testosterone per dose.

In various embodiments, the dose of testosterone ester in the administered oral pharmaceutical composition is increased by the equivalent of about 25 to about 75 mg of testosterone when the plasma testosterone C_{avg} in the subject is less than about 350 ng/dL, and decreased by the equivalent of about 10 to about 75 mg of testosterone when the plasma testosterone C_{avg} in the subject is greater than about 800 ng/dL.

In an embodiment, the dose of testosterone ester in the administered oral pharmaceutical composition is increased by the equivalent of about 40 to about 60 mg of testosterone when the plasma testosterone C_{avg} in the subject is less than about 350 ng/dL.

In an embodiment, the dose of testosterone ester in the administered oral pharmaceutical composition is increased by the equivalent of about 50 mg of testosterone when the plasma testosterone C_{avg} in the subject is less than about 350 ng/dL.

In an embodiment, the dose of testosterone ester in the administered oral pharmaceutical composition is decreased by the equivalent of about 10 to about 60 mg of testosterone when the plasma testosterone C_{avg} in the subject is greater than about 800 ng/dL.

In an embodiment, the dose of testosterone ester in the administered oral pharmaceutical composition is decreased by the equivalent of about 25 to about 50 mg of testosterone when the plasma testosterone C_{avg} in the subject is greater than about 800 ng/dL.

In an embodiment, the dose of testosterone ester in the administered oral pharmaceutical composition is decreased by the equivalent of about 25 mg of testosterone when the plasma testosterone C_{avg} in the subject is greater than about 800 ng/dL.

In various embodiments, the oral pharmaceutical composition is administered twice daily (BID).

In an embodiment, the oral pharmaceutical composition is administered three times daily (TID).

In various embodiments, the oral pharmaceutical composition is administered once daily (QD).

In an embodiment, the oral pharmaceutical composition comprises testosterone undecanoate and is administered twice daily (BID).

In an embodiment, the oral pharmaceutical composition comprises testosterone undecanoate and is administered three times daily (TID).

In an embodiment, the oral pharmaceutical composition comprises testosterone tridecanoate and is administered once daily (QD).

In various embodiments, the plasma testosterone C_{avg} is measured three to six hours after administering the oral pharmaceutical composition.

In another embodiment, the plasma testosterone C_{avg} is estimated on the basis of a single blood sample.

Effective off-diagonal titration can be estimated for the following reasons. First, testosterone exposure is dose proportional, so it is possible to predict the change in C_{avg} with change in dose. Second, the titration boundaries (350 to 800 ng/dL) fall within the eugonadal boundaries (252 to 907 ng/dL), and the eugonadal range is wide (3.5 fold) compared to the largest dose increment (33%) or decrement (25%). This means that the dose increments/decrements employed can allow movement within the eugonadal range (e.g. increasing the dose of someone with a C_{avg} of 400 ng/dL will raise the C_{avg} to a maximum of 532 ng/dL [400×1.33]). Similarly, when titration decisions based on C₄ are different from those based on C_{avg}, the outcome will often be a C_{avg} in the eugonadal range. For example, when a patient's C₄ is less than 350 ng/dL (indicating a dose increase is required), but whose C_{avg} is 600 ng/dL (indicating no titration), the impact of titrating based on C₄ is that the C_{avg} will increase but remain in the eugonadal range. The largest dose increase will result in a 33% increase in exposure which, in this case, will raise the C_{avg} to 798 ng/dL. Therefore, despite titration based on C₄, this patient's C_{avg} is not likely to rise above the upper boundary of the eugonadal range. Thus, the titration decision based on C₄ is effectively concordant with that based on C_{avg}, since both titration decisions will result in a patient with a C_{avg} in the eugonadal range. Selected cells in Table 1 indicate a patient's C_{avg} that would result in effective off-diagonal titration. Therefore, when comparing the effectiveness of dose-titration decisions based on C₄ and C_{avg}, both concordance (on-diagonal agreement between C₄ and C_{avg}) and effective off-diagonal agreement must be considered.

An analysis of study data demonstrates that for certain visits, the incidence of appropriate titration decisions (concordant decisions plus effective off-diagonal decisions) was 88.0% and 93.1%, respectively (Table 2). These reflect a concordance of 63.9% and 58.6% and an effective off-diagonal titration decision of 24.1% and 34.5% for Visit 2 and Visit 4b, respectively. This indicates that titration based on C₄ can effectively adjust a patient's dose such that his C_{avg} is in the eugonadal range.

Modeling and simulation were used to confirm that titration decisions based on C₄ are an effective method to adjust patients' doses to maintain testosterone levels in the eugonadal range and avoid high Cₘₐₓ values. Table 3 shows that dose titration based on C_{avg} yields similar efficacy (94.8%) to that when titration is based on C₄ (94.4%). Modeling and simulation were also used to determine whether a 1-hour window around the 4-hour sample collection time would substantially degrade the dose titration decision. Table 3 presents the results of simulations that compare titration decisions based on C_{avg}, C₄, and C₃₋₅. Regardless of which measure titration is based on, the efficacy remains at approximately 95% (much higher than the FDA target of 75%).

TABLE 3
Estimated % of Patients With C_{avg} Within Interval (95% CI)
on Visit 7

| | <252 ng/dL | | 252-907 ng/dL | | >907 ng/dL | |
|---|---|---|---|---|---|---|
| Target at Visit | | | ≥75% | | | |
| C_{avg}-Based | 3.4 | (0.0-8.5) | 94.8 | (88.9-99.0) | 1.8 | (0.0-4.0) |
| Titration | | | | | | |
| Schemes | | | | | | |
| Single Draw | 4.6 | (1.5-8.7) | 94.4 | (89.8-98.0) | 1.0 | (0.0-3.1) |
| Status Sample | | | | | | |
| at Defined | | | | | | |
| Time Point | | | | | | |
| (C₄) | | | | | | |
| Single Draw | 4.7 | (1.0-10.0) | 94.3 | (89.0-98.5) | 1.0 | (0.0-3.0) |
| Status Sample | | | | | | |
| in Window | | | | | | |
| (C₃₋₅) | | | | | | |

Estimated % of Patients With Cₘₐₓ Within Interval (95% CI)
on Visit 7

| | ≤1500 ng/dL | | >1800-≤2500 ng/dL | | >2500 ng/dL | |
|---|---|---|---|---|---|---|
| Target at Visit | ≥85% | | ≤5% | | 0% | |
| C_{avg}-Based | 91.3 | (85.5-96.5) | 3.7 | (1.0-8.0) | 0.4 | (0.0-2.0) |
| Titration | | | | | | |
| Schemes | | | | | | |
| Single Draw | 94.1 | (89.8-99.0) | 2.0 | (0.0-4.6) | 0.5 | (0.0-2.1) |
| Status Sample | | | | | | |
| at Defined | | | | | | |
| Time Point | | | | | | |
| (C₄) | | | | | | |
| Single Draw | 93.6 | (86.5-98.0) | 2.3 | (0.0-6.5) | 0.5 | (0.0-2.0) |
| Status Sample | | | | | | |
| in Window | | | | | | |
| (C₃₋₅) | | | | | | |

Abbreviations: C₃₋₅ = concentration 3 to 5 hours after morning dose;
C₄ = concentration 4 hours after morning dose;
C_{avg} = average observed concentration over 24 hours;
CI = confidence interval;
Cₘₐₓ = maximum concentration

A single sample drawn 4 hours after the AM dose can effectively guide dose titration. The titration decision agreement (concordance plus effectiveness of off-diagonal titration decision) between C₄ and C_{avg} was high (88% and 93%) at the 2 titration visits.

Simulation also confirmed that the Status sample collection time had some flexibility, and the results of using a single-time Status sample (C₄) was comparable to using a 2-hour window for the Status sample (C₃₋₅).

In certain embodiments, the plasma testosterone C_{avg} is estimated on the basis of a single blood sample collected 3 to 5 hours after administering the oral pharmaceutical composition.

In certain embodiments, the plasma testosterone C_{avg} is estimated on the basis of a single blood sample collected 4 to 6 hours after administering the oral pharmaceutical composition.

In various embodiments, the plasma testosterone C_{avg} determined based on the measurement of T via a radioimmunoassay, an immunometric assay, or a liquid chromatography tandem mass spectrometry (LC-MS/MS) assay.

In an embodiment, the steady-state plasma testosterone C_{avg} is determined based on the measurement of T in a single blood sample collected about 3 to 6 hours after oral T dose after at least seven days of daily treatment with the oral pharmaceutical composition.

In an embodiment, the steady-state plasma testosterone C_{avg} is determined based on the measurement of T in a single blood sample collected about 3 to 5 hours after oral T dose after at least seven days of daily treatment with the oral pharmaceutical composition.

In an embodiment, the steady-state plasma testosterone C_{avg} is determined based on the measurement of T in a single blood sample collected about 4 to 6 hours after oral T dose after at least seven days of daily treatment with the oral pharmaceutical composition.

In various embodiments, the plasma testosterone C_{avg} is determined after at least 10 to 14 days of daily treatment with the oral pharmaceutical composition.

In certain embodiments, the plasma testosterone C_{avg} is determined after at least 30 days of daily treatment with the oral pharmaceutical composition.

In an embodiment, the dose of oral pharmaceutical composition is measured after 21 days of daily treatment.

In an embodiment, the dose of oral pharmaceutical composition is measured after 56 days of daily treatment.

In an embodiment, the dose of oral pharmaceutical composition is measured after 105 days of daily treatment.

In an embodiment, the dose of oral pharmaceutical composition is titrated after at least 30 days of daily treatment.

In an embodiment, the dose of oral pharmaceutical composition is titrated after 35 days of daily treatment.

In an embodiment, the dose of oral pharmaceutical composition is titrated after at least 60 days of daily treatment.

In an embodiment, the dose of oral pharmaceutical composition is titrated after 70 days of daily treatment.

In various embodiments, the oral pharmaceutical composition is administered in close proximity to a meal (e.g., immediately prior or after a meal, or 15 minutes prior to after a meal or 30 minutes prior to or after a meal) wherein said meal contains at least about 15 g of fat.

In an embodiment, said meal contains at least about 30 g of fat.

In an embodiment, said meal contains at least about 45 g of fat.

In various embodiments, the oral pharmaceutical composition comprises a testosterone ester solubilized in a carrier comprising at least one lipophilic surfactant and at least one hydrophilic surfactant in a total lipophilic surfactant to total hydrophilic surfactant ratio (w/w) falling in the range of about 6:1 to 3.5:1, which composition, upon once- or twice-daily oral administration, provides an average plasma testosterone concentration at steady state falling in the range of about 350 to about 800 ng/dL.

In an embodiment, said composition comprises 15-30% (w/w) of said testosterone ester.

In an embodiment, said composition comprises 15-20% (w/w) of said testosterone ester.

In an embodiment, said composition comprises 18-22% (w/w) of said testosterone ester.

In an embodiment, said composition comprises 25-30% (w/w) of said testosterone ester.

In particular embodiments, the testosterone ester is testosterone undecanoate.

In another embodiment, said testosterone ester is testosterone tridecanoate.

In various embodiments, the oral pharmaceutical composition comprises about 10-20 percent by weight of solubilized testosterone ester, about 5-20 percent by weight of hydrophilic surfactant, about 50-70 percent by weight of lipophilic surfactant; and about 10-15 percent by weight of digestible oil, wherein the oral pharmaceutical composition is free of ethanol.

In certain embodiments, the oral pharmaceutical composition comprises: about 15-20 percent by weight of solubilized testosterone ester, about 5-20 percent by weight of hydrophilic surfactant, about 50-70 percent by weight of lipophilic surfactant; and about 1-10 percent by weight of polyethylene glycol 8000.

In various embodiments, the hydrophilic surfactant exhibits an HLB of 10 to 45.

In certain embodiments, the hydrophilic surfactant is selected from the group consisting of polyoxyethylene sorbitan fatty acid esters, hydrogenated castor oil ethoxylates, polyethylene glycol mono- and di-glycerol esters of caprylic, capric, palmitic and stearic acids, fatty acid ethoxylates, polyethylene glycol esters of alpha-tocopherol and its esters and combinations thereof. In particular embodiments, the hydrophilic surfactant is a hydrogenated castor oil ethoxylate.

In various embodiments, the lipophilic surfactant exhibits an HLB of less than 10. In certain embodiments, the lipophilic surfactant exhibits an HLB of less than 5. In particular embodiments, the lipophilic surfactant exhibits an HLB of 1 to 2.

In various embodiments, the lipophilic surfactant is a fatty acid selected from the group consisting of octanoic acid, decanoic acid, undecanoic acid, lauric acid, myristic acid, palmitic acid, pamitoleic acid, stearic acid, oleic acid, linoleic acid, alpha- and gamma linolenic acid, arachidonic acid, and combinations thereof.

In an embodiment, the lipophilic surfactant is chosen from mono- and/or di-glycerides of fatty acids, such as glyceryl distearate, Imwitor 988 (glyceryl mono-/di-caprylate), Imwitor 742 (glyceryl mono-di-caprylate/caprate), Imwitor 308 (glyceryl mono-caprylate), Imwitor 191 (glyceryl mono-stearate), Softigen 701 (glyceryl mono-/di-ricinoleate), Capmul MCM (glyceryl caprylate/caprate), Capmul MCM(L) (liquid form of Capmul MCM), Capmul GMO (glyceryl mono-oleate), Capmul GDL (glyceryl dilaurate), Maisine (glyceryl mono-linoleate), Peceol (glyceryl mono-oleate), Myverol 18-92 (distilled monoglycerides from sunflower oil) and Myverol 18-06 (distilled monoglycerides from hydrogenated soyabean oil), Precirol ATO 5 (glyceryl palmitostearate) and Gelucire 39/01 (semi-synthetic glycerides, i.e., C₁₂₋₁₈ mono-, di- and tri-glycerides), and combinations thereof.

In various embodiments, the digestible oil is a vegetable oil selected from the group consisting of soybean oil, safflower seed oil, corn oil, olive oil, castor oil, cottonseed oil, arachis oil, sunflower seed oil, coconut oil, palm oil, rapeseed oil, black currant oil, evening primrose oil, grape seed oil, wheat germ oil, sesame oil, avocado oil, almond oil, borage oil, peppermint oil and apricot kernel oil.

In various embodiments, the oral pharmaceutical composition comprises one or more additional therapeutic agents. In certain embodiments, the additional therapeutic agents are selected from the group consisting of a synthetic progestin, an inhibitor of type-I and/or type II 5α-reductase (e.g., finasteride and dutasteride), an inhibitor of CYP3A4, thiazide diuretics, and calcium channel blockers, and combinations thereof. In particular embodiments, the one or more additional therapeutic agents comprises a second testosterone ester.

In an embodiment, said thiazide diuretic is selected from the group consisting of chlorothiazide, chlorthalidone, indapamide, hydrochlorothiazide, methyclothiazide, metolazone.

In an embodiment, said calcium channel blocker is selected from the group consisting of Amlodipine, Diltiazem, Felodipine, Isradipine, Nicardipine, Nifedipine, Nisoldipine, Verapamil.

In various embodiments, the oral pharmaceutical composition is filled into a hard or soft gelatin capsule.

In various embodiments, the oral pharmaceutical composition is a liquid, semi-solid or solid dosage form.

In various embodiments, the oral pharmaceutical composition exhibits a percent (%) in vitro dissolution profile in 5% Triton X-100 solution in phosphate buffer, pH 6.8, indicating release from the composition of substantially all of the solubilized testosterone ester within about 2 hours.

In various embodiments, the oral pharmaceutical composition exhibits a percent (%) in vitro dissolution profile in 5% Triton X-100 solution in phosphate buffer, pH 6.8, indicating release from the composition of substantially all of the solubilized testosterone ester within about 1 hour.

In certain embodiments, the composition is free of monohydric alcohol. In certain embodiments, the monohydric alcohol is chosen from C₂-C₁₈ aliphatic or aromatic alcohol. In particular embodiments, the monohydric alcohol is chosen from ethanol and benzyl alcohol.

In particular embodiments, the oral pharmaceutical composition comprises at least one hydrophilic surfactant comprises Cremophor RH 40 (polyoxyethyleneglycerol trihydroxystearate).

In particular embodiments, the lipophilic surfactant comprises oleic acid.

In particular embodiments, the oral pharmaceutical composition comprises about 18 to 22 percent by weight of a solubilized testosterone undecanoate.

In particular embodiments, the testosterone undecanoate is solubilized in a carrier substantially free of ethanol.

In particular embodiments, the oral pharmaceutical composition comprises 15 to 17 percent by weight of the at least one hydrophilic surfactant.

In particular embodiments, the oral pharmaceutical composition comprises 50 to 55 percent by weight of the at least one lipophilic surfactant.

In particular embodiments, the oral pharmaceutical composition comprises about 19.8 percent by weight of solubilized testosterone undecanoate, about 51.6 percent by weight of oleic acid, about 16.1 percent by weight of polyoxyethylene (40) hydrogenated castor oil, about 10 percent by weight of borage seed oil, about 2.5 percent by weight of peppermint oil, and about 0.03 percent by weight of butylated hydroxytoluene (BHT).

In particular embodiments, each morning and evening dose initially comprises about 237 mg of testosterone undecanoate.

In another embodiment, the oral pharmaceutical composition comprises about 15 percent by weight of testosterone undecanoate, about 63 percent by weight of glyceryl mono-linoleate, about 16 percent by weight of polyoxyethylene (40) hydrogenated castor oil, and about 6 percent by weight of polyethylene glycol having a molecular weight of about 8000 g/mol (PEG 8000).

In another embodiment, the composition comprises 20-30% by weight of testosterone tridecanoate, 40-75% by weight of a fatty acid, 2-20% by weight of mono- and/or di-glycerides of fatty acids, and optionally, up to 10% by weight of a hydrophilic surfactant.

Dietary fat content modulates the bioavailability of oral TU and the associated T response observed following oral TU. Thus, in various embodiments, the oral pharmaceutical composition is administered with a meal wherein said meal contains at least about 15 g of fat.

### BRIEF DESCRIPTION OF THE DRAWINGS

FIG. 1 shows the mean concentration-time profiles for T following a single 316 mg oral TU dose, by sample collection tube type.

### DETAILED DESCRIPTION OF THE INVENTION

### Abbreviations and Definitions

To facilitate understanding of the invention, a number of terms and abbreviations as used herein are defined below as follows:

When introducing elements of the present invention or the particular embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein, is intended to qualify the numerical values that it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “plasma,” as used herein, is intended to mean the liquid component of blood that holds the blood cells in whole blood in suspension; this makes plasma the extracellular matrix of blood cells. It makes up about 55% of the body's total blood volume. It is mostly water (up to 95% by volume), and contains dissolved proteins (6-8%) (i.e.—serum albumins, globulins, and fibrinogen), glucose, clotting factors, electrolytes (Na⁺, Ca²⁺, Mg²⁺, HCO₃⁻, Cl⁻, etc.), hormones, carbon dioxide (plasma being the main medium for excretory product transportation) and oxygen. This is in contrast to blood serum which is blood plasma without clotting factors. Further, plasma is derived from blood that is collected differently than when serum is collected, by allowing the blood to clot prior to centrifugation when collecting serum versus immediate centrifugation when collecting plasma.

### Methods

Certain embodiments as disclosed herein provide methods of treating testosterone deficiency or its symptoms and, in particular, optimize the plasma testosterone concentration during chronic treatment.

The present invention provides methods of administering oral pharmaceutical formulations comprising testosterone esters that provide average steady state plasma levels (concentrations) of testosterone, which fall within a desired “normal” or eugonadal range (i.e., about 250-907 ng/dL) while avoiding the high Cₘₐₓ values that are considered by the United States Food and Drug Administration to be undesirable as summarized in Table 4.

TABLE
Exposure Categories, and Proposed Limits, for T Replacement

| Concentration Range | Percent of Population |
|---|---|
| C_{avg} < 300 ng/dL | <25%* |
| 252 ng/dL ≤ C_{avg} ≤ 907 ng/dL | ≥75% |
| C_{avg} > 1000 ng/dL | <25%* |
| Cₘₐₓ ≤ 1500 ng/dL | ≥85% |
| Cₘₐₓ > 1500 ng/dL | <15% |
| Cₘₐₓ > 1800 ng/dL | <5% |
| Cₘₐₓ > 2500 ng/dL | 0% |

*The patients whose C_{avg} does not fall within the normal range for T can have C_{avg} values either above or below the normal range, but the sum of both populations should not exceed 25%.

For instance, FDA approval targets state that less than 15% of treated subjects may have a Cₘₐₓ value of 1500 ng/dL or greater, and that none may have a Cₘₐₓ value exceeding 2500 ng/dL. Less than 5% of treated subjects may have a Cₘₐₓ value falling in the range of 1800-2500 ng/dL.

Modeling studies suggest that 200 mg BID dosing of T (as a testosterone ester) is likely to have a high success rate in terms of C_{avg} being in the normal range, and Cₘₐₓ concentrations not being excessively high, at least after dose titration, and that over-responders, and most of the under-responders can have their plasma T C_{avg} concentration brought into the normal range without exceeding the Cₘₐₓ limitations noted in the guidelines.

Thus, in various embodiments, the present invention provides a method of treating chronic testosterone deficiency or its symptoms comprising the steps of:
a. administering to a subject in need thereof an initial dose of oral pharmaceutical composition comprising a testosterone ester solubilized in a carrier comprising at least one lipophilic surfactant and at least one hydrophilic surfactant;
b. collecting said subject's blood sample in tubes containing NaF;
c. measuring the NaF-containing plasma testosterone concentration in the subject; and
d. administering an increased dose of the oral pharmaceutical composition to the subject when the NaF-containing plasma testosterone concentration in the subject is less than 350 ng/dL, and administering a decreased dose of the oral pharmaceutical composition to the subject when the plasma testosterone concentration in the subject is greater than 800 ng/dL.

In an embodiment, said plasma testosterone concentration is measured in plasma collected in a tube containing NaF.

The administered oral pharmaceutical compositions comprise a hydrophobic testosterone ester dissolved in a lipophilic surfactant and a hydrophilic surfactant. A lipophilic surfactant as defined herein has a hydrophilic-lipophilic balance (HLB) less than 10, and preferably less than 5. A hydrophilic surfactant as defined herein has an HLB of greater than 10. (HLB is an empirical expression for the relationship of the hydrophilic and hydrophobic groups of a surface-active amphiphilic molecule, such as a surfactant). It is used to index surfactants and its value varies from about 1 to about 45. The higher the HLB, the more water-soluble the surfactant. The compositions are designed to be self-emulsifying drug delivery systems (SEDDS) and iterations thereof such as self-microemulsified drug delivery systems (SMEDDS) and self-nanoemulsified drug delivery systems (SNEDDS) so that a testosterone ester-containing emulsion, microemulsion, nanoemulsion (or dispersion) is formed upon mixing with intestinal fluids in the gastrointestinal tract.

In various embodiments, the testosterone ester is a short-chain (C₂-C₆) or a medium-chain (C₇-C₁₃) fatty acid ester located on the C-17 of the testosterone molecule. In certain embodiments, the testosterone ester is testosterone cypionate, testosterone octanoate, testosterone enanthate, testosterone decanoate, or testosterone undecanoate. In particular embodiments, the testosterone ester is testosterone undecanoate. In another embodiment said testosterone ester is testosterone tridecanoate. For calculation purposes, 1 mg of T is equivalent to: 1.39 mg T-enanthate; 1.58 mg T-undecanoate; 1.43 mg T-cypionate, 1.68 mg T-tridecanoate, and 1.83 mg T-palmitate.

In various embodiments, the lipophilic surfactant exhibits an HLB of less than 10, preferably less than 5, and more preferably, the lipophilic surfactant exhibits an HLB of 1 to 2. Certain lipophilic surfactants suitable in oral compositions of the present invention include fatty acids (C₆-C₂₄, preferably C₁₀-C₂₄, more preferably C₁₄-C₂₄), for example, octanoic acid, decanoic acid, undecanoic acid, lauric acid, myristic acid, palmitic acid, palmitoleic, stearic acid, oleic acid, linoleic acid, alpha- and gamma-linolenic acid, arachidonic acid or combinations thereof. In a particular embodiment, the lipophilic surfactant is oleic acid.

Other suitable lipophilic surfactants include:
Mono- and/or di-glycerides of fatty acids, such as glyceryl distearate, Imwitor 988 (glyceryl mono-/di-caprylate), Imwitor 742 (glyceryl mono-di-caprylate/caprate), Imwitor 308 (glyceryl mono-caprylate), Imwitor 191 (glyceryl mono-stearate), Softigen 701 (glyceryl mono-/di-ricinoleate), Capmul MCM (glyceryl caprylate/caprate), Capmul MCM(L) (liquid form of Capmul MCM), Capmul GMO (glyceryl mono-oleate), Capmul GDL (glyceryl dilaurate), Maisine (glyceryl mono-linoleate), Peceol (glyceryl mono-oleate), Myverol 18-92 (distilled monoglycerides from sunflower oil) and Myverol 18-06 (distilled monoglycerides from hydrogenated soybean oil), Precirol ATO 5 (glyceryl palmitostearate) and Gelucire 39/01 (semi-synthetic glycerides, i.e., C12-18 mono-, di- and tri-glycerides);
Acetic, succinic, lactic, citric and/or tartaric esters of mono- and/or di-glycerides of fatty acids, for example, Myvacet 9-45 (distilled acetylated monoglycerides), Miglyol 829 (caprylic/capric diglyceryl succinate), Myverol SMG (mono/di-succinylated monoglycerides), Imwitor 370 (glyceryl stearate citrate), Imwitor 375 (glyceryl monostearate/citrate/lactate) and Crodatem T22 (diacetyl tartaric esters of monoglycerides);
Propylene glycol mono- and/or di-esters of fatty acids, for example, Lauroglycol (propylene glycol monolaurate), Mirpyl (propylene glycol monomyristate), Captex 200 (propylene glycol dicaprylate/dicaprate), Miglyol 840 (propylene glycol dicaprylate/dicaprate) and Neobee M-20 (propylene glycol dicaprylate/dicaprate);
Polyglycerol esters of fatty acids such as Plurol oleique (polyglyceryl oleate), Caprol ET (polyglyceryl mixed fatty acids) and Drewpol 10.10.10 (polyglyceryl oleate);
Castor oil ethoxylates of low ethoxylate content (HLB<10) such as Etocas 5 (5 moles of ethylene oxide reacted with 1 mole of castor oil) and Sandoxylate 5 (5 moles of ethylene oxide reacted with 1 mole of castor oil;
Acid and ester ethoxylates formed by reacting ethylene oxide with fatty acids or glycerol esters of fatty acids (HLB<10) such as Crodet 04 (polyoxyethylene (4) lauric acid), Cithrol 2MS (polyoxyethylene (2) stearic acid), Marlosol 183 (polyoxyethylene (3) stearic acid) and Marlowet G12DO (glyceryl 12 EO dioleate). Sorbitan esters of fatty acids, for example, Span 20 (sorbitan monolaurate), Crill 1 (sorbitan monolaurate) and Crill 4 (sorbitan mono-oleate);
Transesterification products of natural or hydrogenated vegetable oil triglyceride and a polyalkylene polyol (HLB<10), e.g. Labrafil M1944CS (polyoxyethylated apricot kernel oil), Labrafil M2125CS (polyoxyethylated corn oil) and Gelucire 37/06 (polyoxyethylated hydrogenated coconut);
Alcohol ethyoxylates (HLB<10), e.g. Volpo N3 (polyoxyethylated (3) oleyl ether), Brij 93 (polyoxyethylated (2) oleyl ether), Marlowet LA4 (polyoxyethylated (4) lauryl ether); and
Pluronics, for example, Polyoxyethylene-polyoxypropylene co-polymers and block co-polymers (HLB<10) e.g. Synperonic PE L42 (HLB=8) and Synperonic PE L61 (HLB=3)

In various embodiments, the lipophilic surfactant is glyceryl monolinoleate.

In various embodiments, the hydrophilic surfactant exhibits an HLB of 10 to 45. Hydrophilic surfactants with an HLB value between 10-15 are particularly preferred. A hydrophilic surfactant component may be necessary to achieve desirable dispersability of the formulation in the GI tract and release of the drug. That is, a hydrophilic surfactant, in addition to serving as a secondary solvent, may be required to release the drug from the lipid carrier matrix, or primary solvent. The levels (amounts) of the high HLB surfactant can be adjusted to provide optimum drug release without compromising the solubilization of the active ingredient. In certain embodiments, the hydrophilic surfactant is a polyoxyethylene sorbitan fatty acid ester, hydrogenated castor oil ethoxylate, PEG mono- and di-ester of palmitic and stearic acid, fatty acid ethoxylate, or combinations thereof. In a particular embodiment, the hydrophilic surfactant is a hydrogenated castor oil ethoxylate. In another particular embodiment, the hydrophilic surfactant is Cremophor RH 40 (polyoxyethyleneglycerol trihydroxystearate).

In various embodiments, the oral pharmaceutical composition further includes digestible oil. A digestible oil is defined herein as an oil that is capable of undergoing de-esterification or hydrolysis in the presence of pancreatic lipase in vivo under normal physiological conditions. Specifically, digestible oils may be complete glycerol triesters of medium chain (C₇-C₁₃) or long chain (C₁₄-C₂₂) fatty acids with low molecular weight (up to C₆) mono-, di- or polyhydric alcohols. Some examples of digestible oils for use the oral pharmaceutical composition include: vegetable oils (e.g., soybean oil, safflower seed oil, corn oil, olive oil, castor oil, cottonseed oil, arachis oil, sunflower seed oil, coconut oil, palm oil, rapeseed oil, black currant oil, evening primrose oil, grape seed oil, wheat germ oil, sesame oil, avocado oil, almond, borage, peppermint and apricot kernel oils) and animal oils (e.g., fish liver oil, shark oil and mink oil). In certain embodiments, the digestible oil is a vegetable oil. In certain embodiments, the vegetable oil is soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond oil, borage oil, peppermint oil, apricot kernel oil, or combinations thereof. Particular digestible oils are those with high gamma-linolenic acid (GLA) content such as, black currant oil, primrose oil and borage oil, as well as any other digestible oil that can be enriched in GLA acid through enzymatic processes.

In other embodiments of the present invention, methods and compositions for modulating (i.e., sustaining) the rate of available plasma testosterone by incorporating component(s) that may biochemically modulate (1) testosterone ester absorption, (2) testosterone ester metabolism to testosterone, and/or (3) metabolism of testosterone to dihydrotestosterone (DHT). For example, the inclusion of medium to long chain fatty acid esters can enhance testosterone ester absorption. In this way, more testosterone ester may stave off hydrolysis in the gut and enter the blood stream. In other words, the fatty acid ester may competitively inhibit esterases that would otherwise metabolize the testosterone ester. Examples of other esters or combinations thereof include botanical extracts or benign esters used as food additives (e.g., propylparaben, octylacetate and ethylacetate).

Other components that can modulate testosterone ester absorption include “natural” and synthetic inhibitors of 5α-reductase, which is an enzyme present in enterocytes and other tissues that catalyzes the conversion of T to DHT. Complete or partial inhibition of this conversion may both increase and sustain increased plasma levels of T after oral dosing with testosterone ester while concomitantly reducing plasma DHT levels. Borage oil, which contains a significant amount of the 5α-reductase inhibitor, gamma-linolenic acid (GLA), is an example of a “natural” modulator of testosterone ester metabolism. Other than within borage oil, of course, GLA could be added directly as a separate component of a testosterone ester formulation of the invention. Furthermore, any digestible oil as listed above can be enzymatically enriched in GLA. Many natural inhibitors of 5α-reductase are known in the art (e.g., epigallocatechin gallate, a catechin derived primarily from green tea and saw palmetto extract from berries of the Serenoa repens species, phytosterols and lycopene), all of which may be suitable in the present invention. Non-limiting examples of synthetic 5α-reductase inhibitors suitable for use in the present invention include compounds such as finasteride, dutasteride and the like.

In various embodiments, the oral pharmaceutical composition further includes one or more additional therapeutic agents. In certain embodiments, the agent is a second testosterone ester, a synthetic progestin, an inhibitor of type-I and/or type II 5α-reductase, an inhibitor of CYP3A4, finasteride, dutasteride, thiazide diuretics, and calcium channel blockers, or combinations thereof. In a particular embodiment, the agent is borage oil. In another particular embodiment, the agent is peppermint oil and related substances such as menthol and menthol esters. In another particular embodiment, the agent is a second testosterone ester.

In an embodiment, said thiazide diuretic is selected from the group consisting of chlorothiazide, chlorthalidone, indapamide, hydrochlorothiazide, methyclothiazide, metolazone.

In an embodiment, said calcium channel blocker is selected from the group consisting of Amlodipine, Diltiazem, Felodipine, Isradipine, Nicardipine, Nifedipine, Nisoldipine, Verapamil.

Optional cosolvents suitable with the oral pharmaceutical composition are, for example, water, short chain mono-, di-, and polyhydric alcohols, such as ethanol, benzyl alcohol, glycerol, propylene glycol, propylene carbonate, polyethylene glycol (PEG) with an average molecular weight of about 200 to about 10,000, diethylene glycol monoethyl ether (e.g., Transcutol HP), and combinations thereof. In particular, such cosolvents, especially monohydric alcohols, are excluded altogether. Thus, in various embodiments, the oral pharmaceutical compositions are free of monohydric alcohols. In certain embodiments, the monohydric alcohols are C₂-C₁₈ aliphatic or aromatic alcohols. In particular embodiments, the compositions are free of ethyl or benzyl alcohols.

In particular embodiments, the compositions contain between 0% and 10% (w/w) of polyethylene glycol with an average molecular weight of about 8,000 (PEG-8000). In particular embodiments, the compositions contain between 5% and 10% (w/w) of PEG-8000.

The oral pharmaceutical compositions administered in the present invention are preferably liquid or semi-solid at ambient temperatures. Furthermore, these pharmaceutical compositions can be transformed into solid dosage forms through adsorption onto solid carrier particles, such as silicon dioxide, calcium silicate or magnesium aluminometasilicate to obtain free-flowing powders that can be either filled into hard capsules or compressed into tablets. Hence, the term “solubilized” herein, should be interpreted to describe an active pharmaceutical ingredient (API), which is dissolved in a liquid solution or which is uniformly dispersed in a solid carrier. In addition, sachet type dosage forms can be formed and used. In various embodiments, the oral pharmaceutical composition is filled into a hard or soft gelatin capsule.

In a particular embodiment, the present invention provides a method of treating chronic testosterone deficiency or it symptoms comprising the steps of:
a. administering daily to a subject in need thereof an oral pharmaceutical composition comprising 237 mg of testosterone undecanoate solubilized in a carrier comprising oleic acid, polyoxyethyelene (40) hydrogenated castor oil, borage seed oil, and peppermint oil, twice a day, for a period of at least fourteen days;
b. collecting said subject's blood sample in tubes containing NaF;
c. measuring the plasma testosterone concentration in the subject three to six hours following the daily administration of the oral pharmaceutical composition;
d. increasing the dose of testosterone equivalents administered daily to the subject by 50 mg when the plasma testosterone concentration in the subject is less than 350 ng/dL, and decreasing the dose of testosterone equivalents administered daily to the subject by 25 mg when the plasma testosterone concentration in the subject is greater than 800 ng/dL; and
e. repeating steps a.-d. until the plasma testosterone concentration in the subject is between 350 and 800 ng/dL.

In an embodiment, said plasma testosterone concentration is measured four to six hours following the daily administration of the oral pharmaceutical composition.

Provided herein is a method of treating a population of humans suffering from chronic testosterone deficiency comprising the steps of:
a. administering daily to the subject a dose of an oral pharmaceutical composition comprising a testosterone ester solubilized in a carrier comprising at least one lipophilic surfactant and at least one hydrophilic surfactant;
b. collecting said subject's blood sample in tubes containing NaF;
c. measuring the NaF-containing plasma testosterone concentration in the subject; and
d. increasing the dose of testosterone ester administered in step a. when the measured plasma testosterone concentration in the subject is less than about 350 ng/dL, decreasing each dose of testosterone ester administered in step a. when the measured plasma testosterone concentration in the subject is greater than about 800 ng/dL, and maintaining each dose of testosterone ester administered in step a. when the measured plasma testosterone concentration in the subject is between about 350 ng/dL and about 800 ng/dL,
   wherein, after treatment, less than 25% of the population has a plasma testosterone C_{avg} below 350 ng/dL, less than 25% of the population has a plasma testosterone C_{avg} above 800 ng/dL, and 75% of the population has a plasma testosterone C_{avg} between 350 ng/dL and 800 ng/dL.

Disclosed herein is a method of treating a population of humans suffering from chronic testosterone deficiency comprising the steps of:
a. administering daily to the subject a dose of an oral pharmaceutical composition comprising a testosterone ester solubilized in a carrier comprising at least one lipophilic surfactant and at least one hydrophilic surfactant;
b. collecting said subject's blood sample in tubes containing NaF;
c. measuring the NaF-containing plasma testosterone concentration in the subject; and
d. increasing the dose of testosterone ester administered in step a. when the measured plasma testosterone concentration in the subject is less than about 350 ng/dL, decreasing each dose of testosterone ester administered in step a. when the measured plasma testosterone concentration in the subject is greater than about 800 ng/dL, and maintaining each dose of testosterone ester administered in step a. when the measured plasma testosterone concentration in the subject is between about 350 ng/dL and about 800 ng/dL,
   wherein, after treatment, greater than 85% of the population has a plasma testosterone Cₘₐₓ below 1500 ng/dL, less than 15% of the population has a plasma testosterone Cₘₐₓ above 1500 ng/dL, less than 5% of the population has a plasma testosterone Cₘₐₓ above 1800 ng/dL, and 0% of the population has a plasma testosterone Cₘₐₓ above 2500 ng/dL.

After reading this description, it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

Specific embodiments of the instant invention will now be described in non-limiting examples.

The compositions details of Table 5 (mg/capsule and wt. percentage) are based on an approximate fill weight of 800 mg fill weight per ‘00’ hard gelatin capsule. However, at testosterone-ester amounts less than about 100 mg/capsule, the formulations may be proportionally adjusted for smaller total fill weights that would permit use of smaller hard gelatin capsules (e.g., size ‘0’ or smaller size if needed).

As well, it should be apparent to one of ordinary skill in the art that many, if not all, of the surfactants within a category (e.g., lipophilic, hydrophilic, etc.) may be exchanged with another surfactant from the same category. Thus, while Table 2 lists formulations comprising oleic acid, one of ordinary skill in the art should recognize other lipophilic surfactants (e.g., those listed above) may be suitable as well. Similarly, while Table 5 lists formulations comprising Cremophor RH40 (HLB=13), one of ordinary skill in the art should recognize other hydrophilic surfactants (e.g., those listed above) may be suitable. Borage oil, peppermint oil, BHT, and ascorbyl palmitate may be substituted for chemically similar substances or eliminated.

Particular formulations of TU filled into size “00” capsules in accordance with the present invention are:

Formulation A

| Ingredients | mg/capsule | %, w/w |
|---|---|---|
| Testosterone | 158.3 | 19.8 |
| Undecanoate | | |
| Oleic Acid | 413.1 | 51.6 |
| Cremophor RH 40 | 128.4 | 16.1 |
| Borage Seed Oil | 80.0 | 10 |
| Peppermint Oil | 20.0 | 2.5 |
| BHT | 0.2 | 0.03 |
| Total | 800 | 100 |

Formulation B

| Ingredients | mg/capsule | %, w/w |
|---|---|---|
| Testosterone | 158.3 | 19.8 |
| Undecanoate | | |
| Oleic Acid | 412.5 | 51.6 |
| Cremophor RH 40 | 128.4 | 16.0 |
| Peppermint Oil | 20.0 | 2.5 |
| Borage Seed Oil + | 80.0 | 10 |
| 0.03% BHT | | |
| Ascorbyl Palmitate | 0.8 | 0.1 |
| Total | 800 | 100 |

Formulation C

| Ingredients | mg/capsule | %, w/w |
|---|---|---|
| Testosterone | 120 | 15 |
| Undecanoate | | |
| Cremophor RH | 128 | 16 |
| 40 | | |
| Maisine 35-1 | 504 | 63 |
| Polyethylene | 48 | 6 |
| Glycol 8000 | | |
| TOTAL | 800 | 100 |

Table 6 provides composition details of various formulations of testosterone tridecanoate (TT) in accordance with the teachings of the instant invention.

TABLE 6

| | | Labrafil | Precirol | Cremophor | |
|---|---|---|---|---|---|
| ID | TT | M1944CS | AT05 | RH40 | Labrasol |
| A | 400 | 109.68 | 66.49 | 223.83 | — |
| | 50.00% | 13.71% | 8.31% | 27.98% | — |
| B | 360 | 120.64 | 73.14 | 246.21 | — |
| | 45.00% | 15.08% | 9.14% | 30.78% | — |
| C | 320 | 131.61 | 79.79 | 268.60 | — |
| | 40.00% | 16.45% | 9.97% | 33.57% | — |
| D | 280 | 142.58 | 86.44 | 290.98 | — |
| | 35.00% | 17.82% | 10.80% | 36.37% | — |
| E | 240 | 153.55 | 93.09 | 313.36 | — |
| | 30.00% | 19.19% | 11.64% | 39.17% | — |
| F | 228.32 | 156.75 | 95.03 | 319.9 | — |
| | 28.54% | 19.59% | 11.88% | 39.99% | — |
| G | 200 | 164.52 | 99.74 | 335.75 | — |
| | 25.00% | 20.56% | 12.47% | 41.97% | — |
| H | 160 | 175.48 | 106.39 | 358.13 | — |
| | 20.00% | 21.94% | 13.30% | 44.77% | — |
| I | 120 | 186.45 | 113.04 | 380.51 | — |
| | 15.00% | 23.31% | 14.13% | 47.56% | — |
| J | 80 | 197.42 | 119.69 | 402.90 | — |
| | 10.00% | 24.68% | 14.96% | 50.36% | — |
| K | 40 | 208.39 | 126.33 | 425.28 | — |
| | 5.00% | 26.05% | 15.79% | 53.16% | — |
| L | 20 | 213.87 | 129.66 | 436.47 | — |
| | 2.50% | 26.73% | 16.21% | 54.56% | — |
| M | 400 | 199.97 | 66.62 | 133.40 | — |
| | 50.00% | 25.00% | 8.33% | 16.68% | — |
| N | 360 | 219.97 | 73.29 | 146.74 | — |
| | 45.00% | 27.50% | 9.16% | 18.34% | — |
| O | 320 | 239.97 | 79.95 | 160.08 | — |
| | 40.00% | 30.00% | 9.99% | 20.01% | — |
| P | 280 | 259.96 | 86.61 | 173.42 | — |
| | 35.00% | 32.50% | 10.83% | 21.68% | — |
| Q | 240 | 279.96 | 93.27 | 186.76 | — |
| | 30.00% | 35.00% | 11.66% | 23.35% | — |
| R | 228.32 | 285.8 | 95.22 | 190.66 | — |
| | 28.54% | 35.73% | 11.90% | 23.83% | — |
| S | 200 | 299.96 | 99.94 | 200.10 | — |
| | 25.00% | 37.49% | 12.49% | 25.01% | — |
| T | 160 | 319.96 | 106.60 | 213.45 | — |
| | 20.00% | 39.99% | 13.32% | 26.68% | — |
| U | 120 | 339.95 | 113.26 | 226.79 | — |
| | 15.00% | 42.49% | 14.16% | 28.35% | — |
| V | 80 | 359.95 | 119.92 | 240.13 | — |
| | 10.00% | 44.99% | 14.99% | 30.02% | — |
| W | 40 | 379.95 | 126.59 | 253.47 | — |
| | 5.00% | 47.49% | 15.82% | 31.68% | — |
| X | 20 | 389.95 | 129.92 | 260.14 | — |
| | 2.50% | 48.74% | 16.24% | 32.52% | — |
| AA | 400 | 109.79 | 66.55 | 149.72 | 73.94 |
| | 50.00% | 13.72% | 8.32% | 18.72% | 9.24% |
| BB | 360 | 120.77 | 73.21 | 164.69 | 81.33 |
| | 45.00% | 15.10% | 9.15% | 20.59% | 10.17% |
| CC | 320 | 131.75 | 79.87 | 179.66 | 88.72 |
| | 40.00% | 16.47% | 9.98% | 22.46% | 11.09% |
| DD | 280 | 142.73 | 86.52 | 194.64 | 96.12 |
| | 35.00% | 17.84% | 10.82% | 24.33% | 12.01% |
| EE | 240 | 153.70 | 93.18 | 209.61 | 103.51 |
| | 30.00% | 19.21% | 11.65% | 26.20% | 12.94% |
| FF | 228.32 | 156.91 | 95.12 | 213.98 | 105.67 |
| | 28.54% | 19.61% | 11.89% | 26.75% | 13.21% |
| GG | 200 | 164.68 | 99.83 | 224.58 | 110.90 |
| | 25.00% | 20.59% | 12.48% | 28.07% | 13.86% |
| HH | 160 | 175.66 | 106.49 | 239.55 | 118.30 |
| | 20.00% | 21.96% | 13.31% | 29.94% | 14.79% |
| II | 120 | 186.64 | 113.14 | 254.52 | 125.69 |
| | 15.00% | 23.33% | 14.14% | 31.82% | 15.71% |
| JJ | 80 | 197.62 | 119.80 | 269.50 | 133.09 |
| | 10.00% | 24.70% | 14.97% | 33.69% | 16.64% |
| KK | 40 | 208.60 | 126.45 | 284.47 | 140.48 |
| | 5.00% | 26.07% | 15.81% | 35.56% | 17.56% |
| LL | 20 | 214.09 | 129.78 | 291.95 | 144.18 |
| | 2.50% | 26.76% | 16.22% | 36.49% | 18.02% |
| MM | 400 | 81.62 | 94.47 | 223.91 | — |
| | 50.00% | 10.20% | 11.81% | 27.99% | — |
| NN | 360 | 89.78 | 103.92 | 246.30 | — |
| | 45.00% | 11.22% | 12.99% | 30.79% | — |
| OO | 320 | 97.94 | 113.37 | 268.69 | — |
| | 40.00% | 12.24% | 14.17% | 33.59% | — |
| PP | 280 | 106.10 | 122.81 | 291.08 | — |
| | 35.00% | 13.26% | 15.35% | 36.39% | — |
| QQ | 240 | 114.27 | 132.26 | 313.47 | — |
| | 30.00% | 14.28% | 16.53% | 39.18% | — |
| RR | 228.32 | 116.65 | 135.02 | 320.01 | — |
| | 28.54% | 14.58% | 16.88% | 40.00% | — |
| SS | 200 | 122.43 | 141.71 | 335.86 | — |
| | 25.00% | 15.30% | 17.71% | 41.98% | — |
| TT | 160 | 130.59 | 151.16 | 358.25 | — |
| | 20.00% | 16.32% | 18.89% | 44.78% | — |
| UU | 120 | 138.75 | 160.60 | 380.64 | — |
| | 15.00% | 17.34% | 20.08% | 47.58% | — |
| VV | 80 | 146.91 | 170.05 | 403.04 | — |
| | 10.00% | 18.36% | 21.26% | 50.38% | — |
| WW | 40 | 155.08 | 179.50 | 425.43 | — |
| | 5.00% | 19.38% | 22.44% | 53.18% | — |
| XX | 20 | 159.16 | 184.22 | 436.62 | — |
| | 2.50% | 19.89% | 23.03% | 54.58% | — |

Tables 7-9 provides composition details of various TT formulations in accordance with the teachings of the instant invention.

TABLE 8

| | Composition (mg/capsule and weight %) | | | | | | | | |
|---|---|---|---|---|---|---|---|---|---|
| | | | | | Capmul | | | | |
| Formula | | | Cremophor | Oleic | MCM | Tween | Precirol | Gelucire | Fill Wt., |
| No. | TT | Labrasol | RH40 | Acid | (L) | 80 | ATO 5 | 39/01 | mg |
| 27 | 320.0 | — | 240.0 | 220.0 | — | — | 20.0 | — | 800 |
| | 40.0% | | 30.0% | 27.5% | | | 2.5% | | |
| 28 | 364.0 | — | 160.0 | 80.0 | 176.0 | — | 20.0 | — | 800 |
| | 45.5% | | 20.0% | 10.0% | 22.0% | | 2.5% | | |
| 29 | 320.0 | 160.0 | — | — | 300.0 | — | — | 20.0 | 800 |
| | 40.0% | 20.0% | | | 37.5% | | | 2.5% | |
| 30, 34 | 120.0 | — | — | — | 680.0 | — | — | — | 800 |
| | 15.0% | | | | 85.0% | | | | |
| 31, 35 | 120.0 | — | — | — | 560.0 | 120.0 | — | — | 800 |
| | 15.0% | | | | 70.0% | 15.0% | | | |
| 32 | 228.0 | — | 296.0 | 80.0 | 176.0 | — | 20.0 | — | 800 |
| | 28.5% | | 37.0% | 10.0% | 22.0% | | 2.5% | | |
| 33 | 228.0 | 240.0 | — | — | 312.0 | — | — | 20.0 | 800 |
| | 28.5% | 30.0% | | | 39.0% | | | 2.5% | |
| 36 | 120.0 | — | 300.0 | 120.0 | 240.0 | — | 20.0 | — | 800 |
| | 15.0% | | 37.5% | 15.0% | 30.0% | | 2.5% | | |
| 37 | 120.0 | 300.0 | — | — | 360.0 | — | — | 20.0 | 800 |
| | 15.0% | 37.5% | | | 45.0% | | | 2.5% | |
| 38 | 176.0 | — | — | — | 624.0 | — | — | — | 800 |
| | 22.0% | | | | 78.0% | | | | |
| 39 | 228.0 | — | — | — | 572.0 | — | — | — | 800 |
| | 28.5% | | | | 71.5% | | | | |
| 40 | 176.0 | — | — | — | 504.0 | 120.0 | — | — | 800 |
| | 22.0% | | | | 63.0% | 15.0% | | | |
| 41 | 176.0 | — | 120.0 | — | 504.0 | — | — | — | 800 |
| | 22.0% | | 15.0% | | 63.0% | | | | |
| 42, 48 | 176.0 | 120.0 | — | — | 504.0 | — | — | — | 800 |
| | 22.0% | 15.0% | | | 63.0% | | | | |
| 43 | 120.0 | 680.0 | — | — | — | — | — | — | 800 |
| | 15.0% | 85.0% | | | | | | | |
| 44 | 120.0 | 340.0 | — | — | 320.0 | — | — | 20.0 | 800 |
| | 15.0% | 42.5% | | | 40.0% | | | 2.5% | |
| 45 | 120.0 | — | — | 680.0 | — | — | — | — | 800 |
| | 15.0% | | | 85.0% | | | | | |
| 46 | 120.0 | — | 680.0 | — | — | — | — | — | 800 |
| | 15.0% | | 85.0% | | | | | | |
| 47 | 120.0 | — | 660.0 | — | — | — | — | 20.0 | 800 |
| | 15.0% | | 82.5% | | | | | 2.5% | |
| 49 | 120.0 | — | — | 408.0 | 272.0 | — | — | — | 800 |
| | 15.0% | | | 51.0% | 34.0% | | | | |
| 50 | 120.0 | — | — | 370.5 | 309.5 | — | — | — | 800 |
| | 15.0% | | | 46.3% | 38.7% | | | | |
| 51 | 120.0 | 140.0 | — | — | 520.0 | — | — | 20.0 | 800 |
| | 15.0% | 17.5% | | | 65.0% | | | 2.5% | |
| 52 | 182.7 | 97.3 | — | — | 520.0 | — | — | — | 800 |
| | 22.8% | 12.2% | | | 65.0% | | | | |
| 53 | 182.7 | — | 97.3 | 208.0 | 312.0 | — | — | — | 800 |
| | 22.8% | | 12.2% | 26.0% | 39.0% | | | | |
| 54 | 120.0 | — | — | 204.0 | 476.0 | — | — | — | 800 |
| | 15.0% | | | 25.5% | 59.5% | | | | |
| 55 | 182.7 | — | — | 185.2 | 432.1 | — | — | — | 800 |
| | 22.8% | | | 23.2% | 54.0% | | | | |
| 56 | 182.7 | — | — | 536.0 | 81.3 | — | — | — | 800 |
| | 22.8% | | | 67.0% | 10.2% | | | | |
| 59 | 120.0 | — | 320.0 | — | 340.0 | — | — | 20.0 | 800 |
| | 15.0% | | 40.0% | | 42.5% | | | 2.5% | |

A particular formulation of TT in accordance with the present invention is:

| Component | mg/capsule | %, w/w |
|---|---|---|
| Testosterone tridecanoate | 228.32 | 28.5 |
| Cremophor ® RH40 | 320.45 | 40.0 |
| Labrafil ® M 1944 CS | 157.02 | 19.6 |
| Precirol ® ATO 5 | 95.20 | 11.9 |
| Total: | 800 | 100.0 |

Other components that can modulate T-ester absorption include “natural” and synthetic inhibitors of 5α-reductase, which is present in enterocytes and catalyze the conversion of T to DHT. Complete or partial inhibition of this conversion may both increase and sustain increases plasma levels of T after oral dosing with T-esters while concomitantly reducing plasma DHT levels. Borage oil, which contains a significant amount of the 5α-reductase inhibitor gamma-linoleic acid (GLA), is an example of a “natural” modulator of T-ester metabolism. Other than within borage oil, of course, GLA could be directly added as a separate component of TT formulations described herein. Many natural inhibitors of 5α-reductase are known in the art (e.g., epigallocatechin gallate, a catechin derived primarily from green tea and saw palmetto extract from berries of the Serenoa repens species), all of which may be suitable in the present invention. Non-limiting examples of synthetic 5α-reductase inhibitors suitable in the present invention include finasteride and dutasteride.

In addition to 5α-reductase inhibitors, the present invention contemplates the use of inhibitors of T metabolism via other mechanisms. One such point of inhibition may be the cytochrome P450 isozyme CYP3A4 that is present in enterocytes and in liver cells and thus capable of metabolizing testosterone. Accordingly, formulations of the present invention, in some embodiments, include peppermint oil, which is known to contain factors capable of inhibiting CYP3A4.

In yet another embodiment of the present invention, drug delivery systems disclosed herein may also be suitable for ameliorating some of the side-effects of certain strategies for male contraception. For example, progestin-based male contraception substantially suppresses luteinizing hormone (LH) and follicle-stimulating hormone (FSH), and thereby suppresses spermatogenesis, resulting in clinical azoospermia (defined as less than about 1 million sperm/ml semen for 2 consecutive months). However, administration of progestins also has the undesirable side-effect of significantly reducing steady-state plasma testosterone levels.

In such situations, for example, it may be preferable to provide preparations of progestin concomitantly with testosterone or a testosterone derivative (e.g., TU). More preferably, a pharmaceutical preparation according to the invention is provided, comprising progestin—in an amount sufficient to suppress LH and FSH production—in combination with testosterone. In some embodiments, the pharmaceutical preparation is for once-daily, oral delivery.

Drug delivery systems, in one aspect of the present invention, afford the flexibility to achieve desirable pharmacokinetic profiles. Specifically, the formulations can be tailored to deliver medicament in a relatively early peak plasma concentration (Tₘₐₓ) or one that appears later. Similarly, the formulations may be tailored to have a relative steep or wide drop in drug plasma concentration upon obtaining Tₘₐₓ. Accordingly, pharmaceutical preparations of the instant invention may be administered once-daily, twice-daily, or in multiple doses per day, depending on, for example, patient preference and convenience.

One way in which the formulations may be modified to affect these changes is to calibrate the ratio of lipophilic surfactants. The magnitude and timing of the Tₘₐₓ, for example, can be affected by not only the type of lipids used, but also the ratios thereof. For example, to obtain a relatively early Tₘₐₓ, or fast release of the medicament from the delivery system, the concentration of the “controlled-release” lipophilic surfactant (e.g., Precirol) may be reduced relative to the concentration of the other lipophilic solvents (e.g., Labrafil M1944CS). On the other hand, to achieve a delayed Tₘₐₓ, the percentage of “controlled-release” lipophilic surfactant in composition can be increased.

Without being bound by or limited to theory, it is believed that the inventive formulations described herein, in one aspect, enhance absorption of a medicament therein by the intestinal lymphatic system. In this way, drug delivery systems of the present invention can provide extended release formulations that can deliver testosterone into the plasma over several hours. The plasma half-life of testosterone in men is considered to be in the range of 10 to 100 minutes, with the upper range for testosterone administered in a form (i.e., TU) that favors lymphatic absorption. However, oral dosages of the present invention can be taken by a patient in need of testosterone therapy once every about twelve hours to maintain desirable levels of plasma testosterone. In another embodiment, oral dosages are taken by a patient in need of testosterone therapy once every about twenty-four hours. In general, “desirable” testosterone levels are those levels found in a human subject characterized as not having testosterone deficiency.

### Baseline T Concentrations

Baseline concentrations of T were determined prior to the start of the study and immediately prior to the start of each treatment cycle (i.e., after each 7 to 14-day washout period). The washout periods were sufficiently long to assure that T concentrations from the previous dosing cycle were no longer detectable.

### Example—Influence of Blood Collection Tubes

Testosterone undecanoate is metabolized into testosterone. Its degradation in whole blood into testosterone has been studied in conditions typically used in clinical trials. It was observed that TU degrades extensively to testosterone in human blood under conditions typical of harvesting serum, causing overestimation of testosterone concentration.

Historically, most testosterone monitoring for diagnostic purposes and for testosterone replacement therapy (TRT) dose titration has been based on the testosterone concentration in blood concentrations in tubes without additives. For subjects receiving oral testosterone undecanoate (TU), it has been proposed that monitoring of blood concentrations should be done with tubes that contain a nonspecific esterase inhibitor, sodium fluoride (NaF). Collecting the blood samples in tubes containing NaF may influence the blood testosterone concentration. Namely, use of NaF when oral TU is administered will enable a more accurate assay of true circulating T concentration.

Each study participant received a single oral TU dose containing 316 mg TU immediately prior to a standardized breakfast meal comprised of 800 to 1000 calories containing approximately 30 g of fat or about 25 to 30 percent fat. Subjects were instructed to consume the entire breakfast meal in no more than 30 minutes. Blood samples were collected 30 minutes prior to oral TU administration and at 0 (pre-dose), and 1, 2, 3, 4, 5, 6, 7, 8, 9, 10, 11 and 12 hrs post-dose. Because of the timed sample collections, all except for one subject entered the clinical research unit around 7 AM and spend the study period of 12 to 14 hours in the unit.

Blood samples were collected into four different types of tubes; 4 mL Plain tubes, containing no additives, 2 mL tubes containing 3 mg NaF+6 mg sodium EDTA (NaF/EDTA), and 4 mL tubes containing 10 mg NaF+8 mg potassium oxalate (NaF/Ox) at the specified time points. Samples were also collected into 7 mL tubes containing 30 mg NaF, but only at 5 of the specified time points (0, 2, 3, 5, 7 hours). In addition, at least 5 mL of saliva was collected into clean sterile cups for the purpose of measuring salivary T concentrations at predose, and at 3 and 6 hours post dose, which is the anticipated period of TU Tₘₐₓ time.

Assessments completed were assays for T, DHT, TU and DHTU. For each subject, a total of 14 blood samples in red (Plain) top tubes (4 mL/sample), in two gray (NaF/EDTA) top tubes (2 mL/tube into two tubes) and in gray (NaF/Ox) top tubes (4 mL/sample) were collected for analysis of total serum T, DHT, TU and DHTU. A total of 5 blood samples in light gray (NaF) tubes (7 mL/sample) were collected during the course of the blood collection.

The in vitro conversion of TU to T in whole blood samples at ambient temperature was investigated by harvesting approximately 50 mL of blood from subjects prior to their oral TU administration. Aliquots of the pretreatment whole blood were added to 5 Plain sample collection tubes each tube previously spiked with 1 of 5 selected concentrations of TU (0, 30, 100, 300 and 1000 ng/mL). After gentle mixing, the tubes were stored at room temperature (about 23° C.) for 30 minutes prior to separating serum. The resulting serum samples were then frozen at −20° C. pending assay for T, DHT, TU and DHTU concentrations.

The study site was given specific instructions on sample handling. The handling instructions were specific to the sample collection tube type and are presented in the following paragraphs.

Plain Collection Tube (Serum): Blood samples collected in tubes were kept at room temperature for 30 minutes then centrifuged at 4° C. for 20 minutes at >1000 g. For each blood collection tube, serum was separated promptly after centrifugation and equal quantities of serum were transferred into 2 appropriately labelled polypropylene tubes. The 2 identical sets of serum samples were then transferred to the assay laboratory for storage at −20° C. (±5° C.) prior to analysis.

NaF Tubes (Serum): Blood samples collected in tubes were immediately kept on ice for 30 minutes then centrifuged at 4° C. for 20 minutes at >1000 g. For each blood collection tube, serum was separated promptly after centrifugation and equal quantities of serum were transferred into 2 appropriately labelled polypropylene tubes. The 2 identical sets of serum samples were then transferred to the assay laboratory for storage at −20° C. (±5° C.) prior to analysis.

NaF+Oxalate Tubes (Plasma): Blood samples collected in tubes were immediately kept on ice for 30 minutes then centrifuged at 4° C. for 20 minutes at >1000 g. For each blood collection tube, plasma was separated promptly after centrifugation and equal quantities of plasma were transferred into 2 appropriately labelled polypropylene tubes. The 2 identical sets of plasma samples were then transferred to the assay laboratory for storage at −20° C. (±5° C.) prior to analysis.

NaF+EDTA Tubes (Plasma): Blood samples collected in tubes were immediately kept on ice for 30 minutes then centrifuged at 4° C. for 20 minutes at >1000 g. For each blood collection tube, plasma was separated promptly after centrifugation and equal quantities of plasma were transferred into 2 appropriately labelled polypropylene tubes. The 2 identical sets of plasma samples were then transferred to the assay laboratory for storage at −20° C. (±5° C.) prior to analysis.

The frozen samples were stored at −20° C. until assay. All samples from a single participant were analyzed in the same assay run. Serum and plasma from the different tubes were measured using the methods developed for serum. Both T and DHT were measured in the same assay and both TU and DHT were analyzed in the same assay developed for serum measurements. On the day of each assay, the frozen samples were thawed and then assayed for T, and DHT, TU, and DHTU. The time allowed for sample thawing was between 1 to 3 hours. The analyte stability at room temperature was validated for each analyte (Bench Top Stability). Both T and DHT and TU and DHTU were extracted using liquid/liquid extraction where the analyte was purified before analysis by liquid chromatography/tandem mass spectrometry (LC-MS/MS). Each of the methods was validated according to the requirement for bioanalytical analysis.

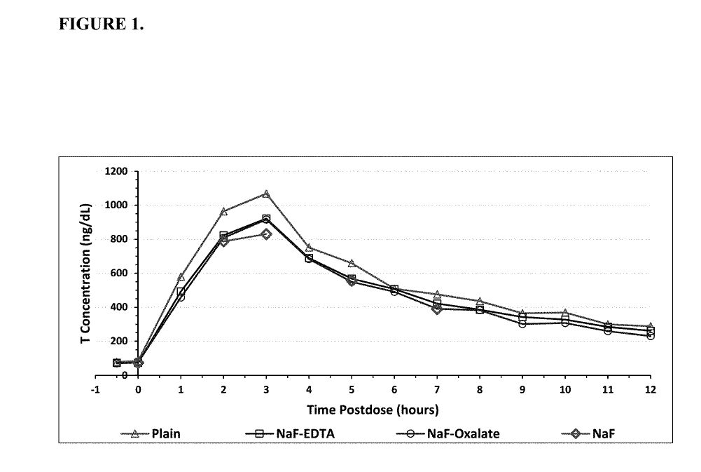

Concentrations of T were assayed at 14 sample collection times over a 12.5-hour period starting 0.5 hours before the oral TU dose was administered and ending 12 hours after dose administration. Mean T concentrations as determined in the Plain tubes are distinctly greater than the profiles for the other 3 types of sample collection tubes. The profiles for the other 3 types of sample collection tubes are clustered together. (The profile for the NaF alone tubes is not a complete set of line segments since data were collected only at the 0, 2, 3, 5 and 7 hour collection times.) The difference between the Plain tube type and the other tube types is greatest at and just prior to the time of peak T concentrations; being approximately 150 ng/dL at 3 hours post dose, the NaF tube types having a mean assayed T concentration approximately 14% less than the Plain tube type (FIG. 1). As expected, the timing of this maximum difference coincides with when TU concentrations are at or near their peak values between 2 and 3 hours post-dose.

Results of the in vivo study showed that, consistent with the hypothesis that de-esterification of TU to T could continue ex vivo, the assayed values for T did depend on the type of additives included in the blood sample collection tubes. Inclusion of NaF in the sample collection tubes to inhibit the de-esterification reactions resulted, on average, in lower assayed T concentrations and lower values for the PK metrics for peak T exposure (Cₘₐₓ) and total T Exposure (AUC₁₂ and C_{avg}).

When serum is harvested in the absence of enzyme inhibitors, the hydrolysis of TU and DHTU to T and DHT occurs during whole blood collection and processing to serum. The conversion of TU to testosterone is extensive and continues over time in whole blood when no enzyme inhibitors are present. Thus, testosterone must be analyzed in enzyme-inhibited plasma when TU is the administered medication.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description, which do not depart from the spirit or scope of the present inventive discovery. Such modifications are also intended to fall within the scope of the appended claims.

What is claimed is:

1. A method of treating chronic testosterone deficiency in a subject in need thereof comprising the steps of:
a. administering daily to the subject a defined dose of an oral pharmaceutical composition comprising a testosterone ester solubilized in a carrier comprising at least one lipophilic surfactant and at least one hydrophilic surfactant;
b. collecting said subject's blood sample in tubes containing NaF;
c. measuring the NaF-containing plasma testosterone concentration in the subject; and
d. increasing the dose of testosterone ester administered in step a. when the measured plasma testosterone concentration in the subject is less than about 350 ng/dL, decreasing each dose of testosterone ester administered in step a. when the measured plasma testosterone concentration in the subject is greater than about 800 ng/dL, and maintaining each dose of testosterone ester administered in step a. when the measured plasma testosterone concentration in the subject is between about 350 ng/dL and about 800 ng/dL.

2. The method of claim 1, wherein the initial defined dose of testosterone ester in the oral pharmaceutical composition is equivalent to about 150 mg of testosterone.

3. The method of claim 2, wherein the defined dose of testosterone ester in the administered oral pharmaceutical composition is increased by the equivalent of about 25-75 mg of testosterone when the plasma testosterone concentration in the subject is less than about 350 ng/dL.

4. The method of claim 2, wherein the defined dose of testosterone ester in the administered oral pharmaceutical composition is decreased by the equivalent of about 10-75 mg of testosterone when the plasma testosterone concentration in the subject is greater than about 800 ng/dL.

5. The method of claim 1, wherein the oral pharmaceutical composition comprises testosterone undecanoate.

6. The method of claim 5, wherein the initial defined dose of the oral pharmaceutical composition administered comprises about 237 mg of testosterone undecanoate.

7. The method of claim 6, wherein the dose of testosterone undecanoate in the administered oral pharmaceutical composition is increased by about 40 mg to about 80 mg when the measured plasma testosterone concentration in the subject is less than about 350 ng/dL.

8. The method of claim 7, wherein the dose of testosterone undecanoate in the administered oral pharmaceutical composition is increased by about 50 mg when the measured plasma testosterone concentration in the subject is less than about 350 ng/dL.

9. The method of claim 6, wherein the dose of testosterone undecanoate in the administered oral pharmaceutical composition is decreased by about 10 mg to about 40 mg when the measured plasma testosterone concentration in the subject is greater than about 800 ng/dL.

10. The method of claim 9, wherein the dose of testosterone undecanoate in the administered oral pharmaceutical composition is decreased by about 25 mg when the measured plasma testosterone concentration in the subject is greater than about 800 ng/dL.

11. The method of claim 1, wherein the oral pharmaceutical composition is administered twice daily.

12. The method of claim 1, wherein the plasma testosterone concentration is measured three to six hours after administering the oral pharmaceutical composition.

13. The method of claim 1, wherein the plasma testosterone concentration is measured four to six hours after administering the oral pharmaceutical composition.

14. The method of claim 1, wherein steps a.-d. are repeated until the plasma testosterone concentration in the subject is between about 350 and about 800 ng/dL.

15. The method of claim 1, wherein the oral pharmaceutical composition comprises:
a. about 15-20 percent by weight of solubilized testosterone ester;
b. about 5-20 percent by weight of hydrophilic surfactant;
c. about 50-70 percent by weight of lipophilic surfactant; and
d. about 10-15 percent by weight of digestible oil.

16. The method of claim 15, wherein the testosterone ester is testosterone undecanoate.

17. The method of claim 15, wherein the hydrophilic surfactant comprises polyoxyethylene (40) hydrogenated castor oil.

18. The method of claim 15, wherein the lipophilic surfactant comprises oleic acid.

19. The method of claim 1, wherein the oral pharmaceutical composition comprises testosterone undecanoate solubilized in a carrier comprising at least one lipophilic surfactant and at least one hydrophilic surfactant in a total lipophilic surfactant to total hydrophilic surfactant ratio (w/w) falling in the range of about 6:1 to 3.5:1, which composition, upon once- or twice-daily oral administration, provides an average plasma testosterone concentration at steady state falling in the range of about 252 to about 907 ng/dL.

20. The method of claim 19, wherein the oral pharmaceutical composition comprises about 18 to 22 percent by weight of solubilized testosterone undecanoate.

21. The method of claim 1, wherein the oral pharmaceutical composition comprises about 19.8 percent by weight of solubilized testosterone undecanoate, about 51.6 percent by weight of oleic acid, about 16.1 percent by weight of polyoxyethylene (40) hydrogenated castor oil, about 10 percent by weight of borage seed oil, about 2.5 percent by weight of peppermint oil, and about 0.03 percent by weight of butylated hydroxytoluene (BHT).

22. The method of claim 1, wherein each morning and evening dose initially comprises about 237 mg of testosterone undecanoate.

23. The method of claim 1, wherein said oral pharmaceutical composition comprises:
a. 15 percent by weight of solubilized testosterone undecanoate;
b. 16 percent by weight of polyoxyethylene (40) hydrogenated castor oil;
c. 63 percent by weight of glyceryl monolinoleate; and
d. 6 percent by weight of polyethylene glycol 8000.

24. A method of treating a population of humans suffering from chronic testosterone deficiency comprising the steps of:
a. administering daily to the subject a defined dose of an oral pharmaceutical composition comprising a testosterone ester solubilized in a carrier comprising at least one lipophilic surfactant and at least one hydrophilic surfactant;
b. collecting said subject's blood sample in tubes containing NaF;
c. measuring the NaF-containing plasma testosterone concentration in the subject; and
d. increasing the dose of testosterone ester administered in step a. when the measured plasma testosterone concentration in the subject is less than about 350 ng/dL, decreasing each dose of testosterone ester administered in step a. when the measured plasma testosterone concentration in the subject is greater than about 800 ng/dL, and maintaining each dose of testosterone ester administered in step a. when the measured plasma testosterone concentration in the subject is between about 350 ng/dL and about 800 ng/dL,
wherein, after treatment, less than 25% of the population has a plasma testosterone C_{avg} below 252 ng/dL, less than 25% of the population has a plasma testosterone C_{avg} above about 907 ng/dL, and 75% of the population has a plasma testosterone C_{avg} between 252 ng/dL and 907 ng/dL.

25. A method of treating a population of humans suffering from chronic testosterone deficiency comprising the steps of:
a. administering daily to the subject a defined dose of an oral pharmaceutical composition comprising a testosterone ester solubilized in a carrier comprising at least one lipophilic surfactant and at least one hydrophilic surfactant;
b. collecting said subject's blood sample in tubes containing NaF;
c. measuring the NaF-containing plasma testosterone concentration in the subject; and
d. increasing the dose of testosterone ester administered in step a. when the measured plasma testosterone concentration in the subject is less than about 350 ng/dL, decreasing each dose of testosterone ester administered in step a. when the measured plasma testosterone concentration in the subject is greater than about 800 ng/dL, and maintaining each dose of testosterone ester administered in step a. when the measured plasma testosterone concentration in the subject is between about 350 ng/dL and about 800 ng/dL,
wherein, after treatment, greater than 85% of the population has a plasma testosterone Cₘₐₓ below 1500 ng/dL, less than 15% of the population has a plasma testosterone Cₘₐₓ above 1500 ng/dL, less than 5% of the population has a plasma testosterone Cₘₐₓ above 1800 ng/dL, and 0% of the population has a plasma testosterone Cₘₐₓ above 2500 ng/dL.

26. A method of treating chronic testosterone deficiency in a subject in need thereof comprising the steps of:
a. administering daily to the subject a dose of an oral pharmaceutical composition comprising a testosterone ester solubilized in a carrier comprising at least one lipophilic surfactant and at least one hydrophilic surfactant;
b. collecting said subject's blood sample in tubes containing NaF;
c. measuring the NaF-containing plasma testosterone concentration in the subject;
d. increasing the dose of testosterone ester administered in step a. when the measured plasma testosterone concentration in the subject is less than about 350 ng/dL, decreasing each dose of testosterone ester administered in step a. when the measured plasma testosterone concentration in the subject is greater than about 800 ng/dL, and maintaining each dose of testosterone ester administered in step a. when the measured plasma testosterone concentration in the subject is between about 350 ng/dL and about 800 ng/dL; and
e. repeating steps a.-d. until the plasma testosterone concentration in the subject is between about 350 and 800 ng/dL.

27. The method of claim 1 wherein said testosterone concentrations are measured in plasma from a blood sample collected in a NaF-EDTA tube drawn 4-6 hours after the morning dose and at least 7 days after starting treatment and following dose adjustment.
